(19) United States
(12) Patent Application Publication
(10) Pub. No.: US 2005/0171116 A1
(43) Pub. Date: Aug. 4, 2005

(54) Enhancing the efficacy of tubercular drugs

(76) Inventor: Vinay Ramakant Sapte, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/851,601

(22) Filed: May 21, 2004

(30) Foreign Application Priority Data
Feb. 4, 2004 (IN) 124/MUM/2004
May 7, 2004 (IN) 527/MUM/2004

(51) Int. Cl. A61K 31/4965; A61K 31/496; A61K 31/355

(52) U.S. Cl. 514/252.13; 514/255.06; 514/356; 514/458

(57) ABSTRACT

Alpha-tocopherol shows broad-spectrum anti-microbial activity against Mycobacterium Tuberculosis bacilli, against the rifampicin-resistant, isoniazid-resistant and multi-drug-resistant strains of M. tuberculosis, against Pseudomonas, against Staphylococci and against Escherichia coli. Further, the antimicrobial activity of known anti-tubercular pharmaceuticals can be improved by administering the anti-tubercular pharmaceutical together with α-tocopherol.

### RELATED APPLICATIONS

This application is a continuation-in-part of Vinay Ramakant SAPTE, Stabilized Short-Course Chemotherapy (SCC) Anti-Tuberculosis Drug Compositions . . . , U.S. application Ser. No. 10/______ filed May 7, 2004.

### BACKGROUND

My invention relates to improvement in methods for the treatment of pulmonary tuberculosis by conjoint administration of α-tocopherol along with routinely administered anti-tubercular drugs such as rifampicin, isoniazid, pyrazinamide or ethambutol.

Mycobacterium tuberculosis bacilli (MTB) are the most common causative organism for pulmonary tuberculosis (PTB). Studies before the advent of modem anti-tubercular drugs showed that ⅓rd of the patients died within a year after diagnosis, and more within five years. Of the survivors after five years, 60% had undergone spontaneous remission, whereas the remaining 40% still continued to excrete tubercle bacilli.

Even after the advent and use of anti-tubercular drugs, the current scenario (as per World Health Organization) shows that the global epidemic of tuberculosis is growing and becoming more dangerous. Currently, the World Health Organization estimates that there are 2 billion people worldwide (one third of the world's population) infected with the TB bacillus (that is to say, having a latent tuberculosis infection, or LTBI). Five to ten percent of the people who are infected with TB (but who are not infected with HIV) become sick or infectious at some time during their life. The epidemics of HIV/ADS and multi-drug resistant tuberculosis (MDR-TB) have also impacted on the spread of TB.

Tuberculosis is the single most common opportunistic infection for people with HIV. Thus, TB is a major cause of death in people who are HIV positive. It accounts for about 11% of AIDS deaths worldwide. If TB control measures are not further strengthened and present trends continue, it is estimated that between the year 2000 and the year 2020, approximately 1 billion people will be newly-infected with the TB bacillus; 150 million people will get sick with active TB disease; and 36 million people will die from TB disease.

Tuberculosis Infection Etiology

The causative organism MTB is a facultative intracellular parasite that has evolved successful strategies to invade and persist inside macrophages which are first line defense cells in lungs. MTB have number of peculiarities. These organisms are sluggishly motile, slow to multiply, and produce neither exotoxin nor endotoxin (which can cause toxic effects in an infected or diseased person). They have a peculiarly-different cell wall structure when compared with other common microorganisms capable of producing diseases in human populations. The cell wall structure has given these organisms a peculiar resistance to acids washes in staining procedures; these microorganisms are thus often called as Acid-Fast Bacilli (AFB).

Recent developments in the knowledge of structure and chemistry of mycobacterium have demonstrated three structural components: plasma membrane, cell wall and capsule. The innermost structure is plasma membrane which appears to be a typical bacterial membrane, playing little part in the pathological process.

The cell wall is unusual in that it has an unusual layer of lipid (mycolate esters), which forms a permeability barrier to polar substances. In structure, it forms a covalently-linked “cell wall skeleton” associated with numerous non-covalently linked substances.

The outermost-component of the cell consists of a mixture of polysaccharide, protein and lipid; this layer is called a “capsule.” The constituents of the capsule are non-covalently bound to the cell wall, and structurally behave like a capsule.

The envelope has a dynamic structure; in growing mycobacterium, molecules are moving within and through the envelope and contribute to a very stable wall skeleton which is continuously reconstructed.

This peculiar “capsule” determines which constituents of the organism are able to be immunologically detected or “seen” by the host cell. The capsule structure also determines which constituents of the host can reach the inside of mycobacterium, and how readily anti-mycobacterial drugs can penetrate into the interior of TB organism. This capsule thus protects the intracellular TB organism from being killed by the host bactericidal mechanisms during the initial stages of infection. The remarkable resistance of mycobacterium tuberculosis to damage is well known, and is attributable to the remarkable impermeability of the capsule (which defends the TB bacterium from the host's defense mechanisms such as host enzymes, reactive oxygen species or free radicals), to the limited immunological visibility (which modulates the host immune-response), and to the selective permeability (which modulates the processing of host-derived materials for use by mycobacterium).

The sheer size of the population worldwide infected by MTB points to an intimate pathogen-macrophage interaction, which is so successfully governed in favor of MTB due to the TB cell wall's chemical and biological constituents.

In the early phase of infection, recognition of a foreign-material signal by the host is followed by the movement of alveolar non-activated macrophages from the host to the TB infection site. The bacilli are then actively phagocytosed by the alveolar macrophages, where the host's immune system uses proteolytic enzymes, cytokines and free radicals to attempt to destroy the TB bacillus. Free radicals act as tiny missiles, with a binding affinity for many cell membrane constituents and intracellular constituents. A free-radical attack on cell membrane constituents results in cell membrane damage, which can ultimately lead to cell death The cell wall of many common pathogenic organisms show susceptibility to destruction by free radicals. Virulent strains of mycobacterium tuberculosis shows peculiar resistance to damage by free radicals. Nor-virulent strains of MTB are susceptible to destruction by free radicals. On the other hand, the cell membrane of human cells is also susceptible to damage by free radicals.

Usually, this phase of infection ends in the intracellular multiplication of MTB, which eventually destroy the macrophages.

This early stage, a non-specific host response, is followed by a stimulation of the host immune system. This leads to a more-specific cell mediated immune response involving T-lymphocytes, which are stimulated by antigens processed and presented by the alveolar macrophages.

This response has two components. One component is a tissue-damaging response, and other component is a macrophage-activating response. The tissue-damaging response is due to a delayed-type hypersensitivity (DTH) reaction to bacillary antigens; this hypersensitivity destroys non-activated macrophages containing multiplying TB bacilli. The macrophage-activating response is a cell-mediated phenomenon causing activation of macrophages which are capable of king and digesting tubercle bacilli.

The balance between these two responses determines the form of tuberculosis that will develop subsequently. A relatively weaker macrophage-activating response causes extensive tissue destruction; the resulting lesions tend to enlarge further and surrounding tissue is progressively damaged. The central caseous material liquefies; the bronchial wall and blood vessels are invaded and destroyed, leading to cavity formation. The liquefied caseous material containing large number of tubercle bacilli is drained through the bronchi. Within the cavity, tubercle bacilli multiply well and spread into the environment through expectorated sputum.

Activated macrophages kill or inhibit the growth of tubercle bacilli probably by more than one way. There are several evidences to show that H₂O₂ and reactive oxygen intermediates (Superoxide anions, hydroxyl ion, singlet oxygen, etc . . . ), are produced by macrophages during its metabolism, and are detrimental to the TB organism. GATEY et al. have isolated a lymphokine which is capable of increasing the intracellular concentration of hydrogen peroxide. Free radicals or reactive oxygen species are extraordinarily active species of molecules which act as unguided missiles which can react with cell membrane of microorganisms as well as human cells leading to cell membrane damage by peroxidation of polyunsaturated fatty acids and which can eventually lead to cell mutation and/or cell death.

Factor Alpha tx

All currently available anti-tubercular drugs are either bactericidal or bacteriostatic. None of these agents have been shown to possess any antioxidant or immuno-stimulant effect. Factor Alpha tx (commonly known as α-tocopherol) is known to be an agent of use in symptomatic vitamin E deficiency. High doses of actor Alpha tx have been shown to improve parameters of immune function, protect against cardiovascular disease (possibly by inhibiting LDL oxidation), and protect against brochopulmonary displasia and retrolental fibroplasias in premature babies. Factor Alpha tx acts as chain-breaking antioxidant and is an efficient pyroxyl radical scavenger, which protects LDLs and polyunsaturated fatty acids in cell membranes from oxidation. Factor Alpha tx also inhibits prostaglandin synthesis and activities of protein kinase C and phospholipase A2.

Vitamin C is known to be an agent of use in scurvy, idiopathic methhaemoglobinaemia, in high doses to prevent viral respiratory infection and in cancer patients. Vitamin C maintains Factor Alpha tx in a chemically-reduced state, and thereby increases Factor Alpha tx's antioxidant effect in vivo. Factor Alpha tx and vitamin C can thus work in a combination which is complimentary in nature.

The aims and objectives of antitubercular therapy in pulmonary tuberculosis patients, with currently available antitubercular drugs during the intensive phase of therapy are twofold:
1) To convert sputum smear-positive cases (who can spread the disease by airborne droplets) in the minimum possible time period into sputum smear-negative cases, so that further spread of MTB is prevented to non-infected individuals in the society.
2) To kill causative organisms, and so eliminate them from the infected individual and to make him/her disease free.
   The currently-available method of treatment of pulmonary tuberculosis (treatment with an antimicrobial) does not address damage caused to host cells by the increasingly-generated free radicals generated by the host's own immune response, and alteration in a favorable way of suppressed/altered immune system response observed in tuberculosis patients.

I thus set about to find a method of treatment with a more-comprehensive approach to treating the multi-faceted patho-physiological changes which occur during tubercular infection of lungs, achieving bactericidal or bacteriostatic activity efficacy against the tubercle bacilli, together with lessened damage to host tissue cells.

### SUMMARY

My invention improves upon one or more shortcomings or disadvantages in the currently-available treatment for pulmonary tuberculosis through the use of antioxidants. In my currently-preferred embodiments, my invention contemplates the use of antioxidants, and particularly Factor Alpha tx and vitamin C, concomitantly with antimicrobial drugs to treat patients with tuberculosis.

In certain embodiments, my invention concerns a method for treating tuberculosis by the application of a therapeutically-effective or prophylactically-effective dose of antioxidants (preferably from about 200 mg to 1,000 mg per day of Factor Alpha tx, most preferably about 400 mg per day, perhaps also used with vitamin C) to human subjects with the disease. As used herein, the term “therapeutically-effective dose” is used to signify that the compound (e.g., the antioxidant or the anti-tubercular drug) is supplied to the patient in amounts and for a period of time effective to provide improvement in one or all of the clinically-measurable parameters of the disease.

I propose that several other infective and non-infective diseases showing raised levels of oxidative-stress may be treated with concomitant use of antioxidants, as per my invention. These diseases include, for example, infectious hepatitis, rheumatic disease, autoimmune disorders, certain hematological disorders and neoplasm.

To determine whether there has been an improvement in one or more of clinically-measured parameters of the disease, one may determine the value of such a parameter in a given patient both before and during treatment. Various clinical signs and symptoms are known to be suitable by those of skill in the art to be suitable as markers of disease severity. For example, sputum smear AFB, radiological findings and lipid peroxidation levels all afe used as clinical indicators for the TB infected patient.

A peculiar advantage of my invention is that it involves application and use of agents already in use as nutrients, and thus known to be safe. By utilizing substances that are already approved for nutritional use, I have provided safe agents for use in new modified and improved treatment strategies against diseases and disorders involving a nonspecific immune response. Accordingly, antioxidants are considered to be of concomitant use with other suitable therapeutic agent in above diseases.

I propose that the Factor Alpha tx may be administered to the patients in any pharmaceutically acceptable vehicle and by any route heretofore acceptable for these agents and other concomitant medicament. I currently prefer an oral route of administration, although one may, if desired, choose to administer the drugs parenteraly, sublingually, as a sustained-release preparation, or by inhalation.

### DETAILED DESCRIPTION OF THE INVENTION

The novel method of this invention comprises the treatment of patients diagnosed as having pulmonary tuberculosis with antioxidants, in an amount effective against the cellular damage caused by the non-specific immune response, concomitantly with antitubercular drugs. This method comprises administering an effective amount of an antioxidant, concomitantly with antitubercular drugs to patients diagnosed as having pulmonary tuberculosis. Even more particularly, the method of the present investigation comprises administering an effective amount of Factor Alpha tx concomitantly with an antimicrobial drugs to patients with such disease.

To date, there has been no information as to Factor Alpha tx's use in the treatment of tuberculosis either alone or along with other antitubercular agent. I herein disclose that Factor Alpha tx is effective in the treatment of tuberculosis when used concomitantly with other antitubercular drugs. The antioxidants and concomitantly-used antitubercular drugs of the present investigation may be administered orally.

Factor Alpha tx and antitubercular drugs used concomitantly are available commercially. For example, the following are commercially-available forms. All the drugs used were administered orally. Therapeutically-effective daily doses of Factor Alpha tx and antitubercular drugs were administered.

The exact mechanism by which concomitant Factor Alpha tx use exerts beneficial effect in pulmonary tuberculosis patients is unknown. However, I postulate that Factor Alpha tx quenches free radicals liberated, and thereby causes a reduction in cell membrane damage of macrophages and other cells involved in cell mediated immunity. This improved immune cell functions results in increased bactericidal activity by host defense cells. In addition to this, the damage to lung tissue at the site of the lesion by released free radicals is also reduced due to quenching by antioxidants used concomitantly; this may lessen the occurrence of the cavities mentioned above.

The following examples are included to demonstrate my currently-referred embodiments of my invention.

### EXAMPLE I

Trial of antioxidants administered concomitantly with anti-tubercular antimicrobial drugs in patients suffering from pulmonary tuberculosis.

Methods

Fifty four patients suffering from pulmonary tuberculosis were included in this study. Thirty patients were treated with four anti-tubercular drugs only. Twenty-four patients were treated with four anti-tubercular drugs and antioxidant drugs concomitantly.

Four antitubercular drugs were given by oral route to all 54 patients in doses and frequency as shown below for a period of 60 days. The dosage regime for the anti-microbial drugs is known in the art as “2EHRZ.” It is an oral daily capsule of rifampicin, a tablet of isoniazid, a tablet of Pyrizinamide, and a tablet of ethambutal.

Out of the total test population of fifty-four patients, twenty-four patients received, in addition to the above 2EHRZ regime, Factor Alpha tx for 60 days, administered daily, orally.

Chest radiology, sputum smears for AFB and lipid peroxidation values were conducted both before and during the study. The patients were assessed at zero, two, four and eight weeks. Various clinical parameters were measured along with chest radiology, sputum smear for AFB and lipid peroxidation values.

The outcome of clinical improvement was accessed by following parameters

1) Sputum AFB Test

In both of the two groups, three sputum samples for each patient were collected and were analyzed for the presence of AFB after 15 days, after 30 days and after sixty days. Analysis was by the Ziehl-Neelson (hot) stain method. The reports were documented as positive for presence of bacilli & negative for absence of bacilli.

2) Radiological assessment was conducted at time zero and at the end of sixty days. The observation were reported as favorable response (i.e., a Static, Improved, or Clear assessment) or an unfavorable response (i.e., Deterioration).

3) Levels of LPO in terms of MDA-TBA complex nmol/g of Hb

All the patients were routinely checked for symptomatic relief every 15 days during the intensive phase. Body weight & Hb % was measured before and at the end of the studies.

Eligible patients were selected from the TB outpatient department population. To be eligible for enrollment in the study, each patient was first screened to meet the following Inclusion Criteria.

1. Patient must have clinically-definite pulmonary tuberculosis-diagnosed for the first time.
2. Patient should be between 21 and 55 years of age inclusive.
3. Patients should have demonstrable pulmonary lesion on chest radiography.
4. Patient's sputum should be positive for AFB.
   In addition, potential test subjects were screened against the following Exclusion Criteria.
1. Patients having age less than 21 years & more than 55 years.
2. Pregnant females & lactating mothers.
3. Patients with sputum AFB negative & or extra pulmonary tuberculosis.
4. Patients already taking treatment for PTB or previously diagnosed for PTB.
5. Patients of PTB with concomitant diseases like diabetes, asthma, cancer or HIV.
   At the screening visit, after the patient has been informed about all aspects of the study and a detailed history of the patient has been obtained, the patients had a chest x-ray, sputum smear for AFB and 5 ml blood was taken from anticubital vein which was analyzed for hemoglobin % and lipid peroxidation values. The patient also had general examination and respiratory system examination and cardiovascular system examination.

The eligible patients were enrolled into the study and were treated with four antitubercular drugs in daily oral doses mentioned above. This group of patients was randomly divided into two groups.

The first group (the control group) received Rifampicin, Isoniazid, Ethambutol and Pyrazinamide in daily oral doses mentioned above, on Out Patient Department (OPD) basis for 60 days. The second group (the study group) received Vitamin E and Vitamin C oral, daily, as a single dose of an anti-oxidant effective amount, conjointly with the aforementioned four antitubercular drugs on an OPD basis.

Chest x-ray, Sputum smear for AFB, and Lipid peroxidation levels were done on day zero, fifteen, thirty and sixty days on OPD basis.

The results of the study indicated that study group showed better sputum negativity (50% in 30 days) compared to the control group (14% in 30 days). X-ray findings showed better improvement in the study group as compared to the control group, probably indicating lessened lung damage. Levels of oxidative stress, as measured in terms of lipid peroxidation, were reduced in the study group and, in contrast, showed a persistent rise over time in the control group.

Better and earlier control of the spread of tuberculosis, along with lessened lung-tissue damage, appear to be the main benefits for TB patients and for human society at large.

The concurrent administration of Factor Alpha tx with routine anti tubercular agents shows synergistic effects in augmenting sputum negativity, potentially by increasing the bactericidal action of antitubercular agents on one hand, while correcting the oxidant/antioxidant imbalance. With corrected oxidant balance, there is better radiological improvement and less lung damage.

The combination method of Factor Alpha tx administered with antitubercular agents appears to be effective in correcting the drawbacks of current treatment of MTB.

### EXAMPLE II

In vitro testing shows interesting and unexpected antimicrobial activity of Factor Alpha tx when included in a standard in vitro cell culture medium.

The results of a study of the antibacterial efficacy of Factor Alpha tx carried in LJ medium in in vitro cell culture shows that Factor Alpha tx is effective against sensitive strains of Mycobacterium Tuberculosis bacilli (MTB), as well as the Rifampicin-resistant, Isoniazid-Resistant and Multi-Drug-Resistant strains of MTB.

The results of studies conducted in broth and tested by culture growth after 24 hrs shows that the Factor Alpha tx has antibacterial activity against Pseudomonas, against Staphylococci and against Escherichia coli.

It should be appreciated by those of skill in the art that the techniques disclosed in the examples discussed above represent techniques I have found to function well in the practice of my invention, and thus can be considered to constitute my currently-preferred modes for its practice. However, those of skill in the art should, in the light of the present disclosure, appreciate that many changes can be made in the specific embodiments which are disclosed, and still obtain a like or similar result, without departing from the spirit and scope of the invention. I thus intent the coverage of my patent to be defined not by the specific examples discussed above, but rather by the claims I append below, and their legal equivalents.

Note that in the claims I use the term “a” to include one or more than one. Thus, for example, the phrase “an antimicrobial” means one or more antimicrobials. Similarly, the phrase “a drug selected from the group consisting of: A, B and C” means one or more drugs selected from that group (i.e., A alone, or A combined with B, or A combined with B and C, etc . . . ).

The term “conjoint” is used as defined in WEBSTER'S THIRD NEW INTERNATIONAL DICTIONARY (1986) (Merriam-Webster, Inc., publ.), namely, “to joint together (as separate entities) for a common purpose or common end.” The term does not require any physical joining nor physical mixture.

The term “drug” is defined in the Federal Food, Drug & Cosmetic Act; under this definition, α-tocopherol is not a “drug.” Thus, α-tocopherol is not an “antimicrobial drug.”

Reduction and oxidation are terms known in the art, a “reducing amount” is an amount effective to chemically reduce a compound from an oxidized state, or to prevent the oxidation of a reduced compount.

1. A composition of matter comprising:
a. a reducing-effective amount of α-tocopherol, and
b. an anti-microbial effective amount of an anti-microbial drug.

2. The composition of claim 1 in powder form.

3. An article of manufacture comprising the composition of claim 2 contained in an envelope packet.

4. A method of combating infection in a patient, the method comprising
a. diagnosing infection in said patient; and
b. administering to said patient the composition of matter of claim 1.

5. The composition of matter of claim 1, comprising from about 0.2 to about 1.0 grams of α-tocopherol.

6. A method of combating infection in a patient, the method comprising
a. diagnosing infection in said patient; and
b. administering to said patient the composition of matter of claim 5.

7. The composition of claim 1, said anti-microbial drug comprising an anti-microbial drug selected from the group consisting of: rifampacin; isoniazid; pyrazinamide; and ethambutol.

8. A method of combating tuberculosis in a patient, the method comprising:
a. diagnosing tuberculosis in said patient; and
b. administering to said patient the composition of matter of claim 7.

9. The composition of claim 7, said antimicrobial compound comprising more than one anti-microbial drug selected from the group consisting of rifampacin; isoniazid; pyrazinamide; and ethambutol.

10. A method of combating infection in a patient the method comprising:
a. diagnosing infection in said patient; and
b. administering to said patient the composition of matter of claim 9.

11. The composition of claim 8, said anti-microbial drug comprising rifampacin and isoniazid and pyrazinamide and ethambutol.

12. A method of combating infection in a patient, the method comprising
a. diagnosing infection in said patient; and
b. administering to said patient the composition of matter of claim 11.

13. The composition of claim 11 in powder form.

14. An article of manufacture comprising the composition of claim 13 contained in an envelope packet.

15. A method of combating infection in a patient, the method comprising
a. diagnosing infection in said patient; and
b. administering to said patient the composition of matter of claim 14.

16. The composition of claim 7, comprising:
a. from about 0.2 to about 1.0 grams of α-tocopherol and
b. an anti-microbial drug selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

17. The composition of claim 16, comprising:
a. from about 0.2 to about 1.0 grams of α-tocopherol and
b. more than one anti-microbial drug selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

18. The composition of claim 17, comprising:
a. from about 0.2 to about 1.0 grams of α-tocopherol, and
b. from about 60 to about 600 mg of rifampacin; and from about 75 to about 700 mg of isoniazid; and from about 150 to about 1,500 mg of pyrazinamide; and from about 100 to about 1,000 mg of ethambutol.

19. The composition of claim 1, further comprising ascorbic acid in an amount effective to maintain said α-tocopherol in a chemically-reduced state before administration to a patient.

20. A method of combating infection in a patient, the method comprising
a. diagnosing infection in said patient; and
b. administering to said patient the composition of matter of claim 19.

21. The composition of claim 19, said anti-microbial drug comprising an anti-microbial drug selected from the group consisting of rifampacin; isoniazid; pyrazinamide; and ethambutol.

22. The composition of claim 21, said anti-microbial compound comprising more than one antimicrobial drug selected from the group consisting of: rifampacin; isoniazid; pyrazinamide; and ethambutol.

23. The composition of claim 22, said anti-microbial drug comprising rifampacin and isoniazid and pyrazinamide and ethambutol.

24. A method of combating infection in a patient, the method comprising
a. diagnosing infection in said patient; and
b. administering to said patient the composition of matter of claim 23.

25. The composition of claim 23 in powder form.

26. An article of manufacture comprising the composition of claim 25 contained in an envelope packet.

27. A method of combating infection in a patient, the method comprising
a. diagnosing infection in said patient; and
b. administering to said patient the composition of matter of claim 22.

28. The composition of claim 22, comprising:
a. from about 0.2 to about 1.0 grams of α-tocopherol and
b. an anti-microbial drug selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

29. The composition of claim 28, comprising:
a. from about 0.2 to about 1.0 grams of α-tocopherol and
b. more than one anti-microbial drug selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

30. The composition of claim 29, comprising:
a. from about 0.2 to about 1.0 grams of α-tocopherol, and
b. from about 60 to about 600 mg of rifampacin; and from about 75 to about 700 mg of isoniazid; and from about 150 to about 1,500 mg of pyrazinamide; and from about 100 to about 1,000 mg of ethambutol.

31. A method of combating infection in a patient, the method comprising
a. diagnosing infection in said patient; and
b. administering to said patient an anti-microbial effective amount of α-tocopherol.

32. The method of claim 31, wherein said infection comprises tuberculosis.

33. The method of claim 32, wherein said anti-microbial effective amount is from about 0.2 to about 1.0 grams of α-tocopherol.

34. A composition of matter comprising:
a. a reducing-effective amount of α-tocopherol, and
b. an anti-microbial effective amount of an anti-microbial drug in a stable complex with Schardinger Sugar in an amount from about 5% to about 50% (w/w) of said anti-microbial drug.

35. The composition of claim 34, said anti-microbial drug selected from the group consisting of: rifampacin; isoniazid; pyrazinamide; and ethambutol.

36. The composition of claim 35, said antimicrobial drug comprising more than one compound selected from the group consisting of: rifampacin; isoniazid; pyrazinamide; and ethambutol.

37. The composition of claim 36, said anti-microbial drug comprising rifampacin and isoniazid and pyrazinamide and ethambutol.

38. The composition of claim 37 in powder form.

39. An article of manufacture comprising the composition of claim 38 contained in an envelope packet.

40. A method to treat tuberculosis comprising administering the composition of matter of claim 37 to a patient.

41. The composition of claim 36, said anti-microbial drug selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

42. The composition of claim 42, said anti-microbial drug comprising more than one compound selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

43. The composition of claim 42 in powder form.

44. The composition of claim 42, said anti-microbial drug comprising: from about 60 to about 600 mg of rifampacin; from about 75 to about 700 mg of isoniazid; from about 150 to about 1,500 mg of pyrazinamide; and from about 100 to about 1,000 mg of ethambutol.

45. An article of manufacture comprising the composition of claim 44 contained in an envelope packet.

46. A method to treat tuberculosis comprising administering the composition of matter of claim 44 to a patient.
